(19) United States
(12) Patent Application Publication
(10) Pub. No.: US 2021/0043294 A1
(43) Pub. Date: Feb. 11, 2021

(54) COLD SYMPTOM TRACKING AND TREATMENT

(71) Applicant: COLD FAid, LLC, Victor, NY (US)

(72) Inventor: Julie DICKENS, Victor, NY (US)

(73) Assignee: COLD FAid, LLC, Victor, NY (US)

(21) Appl. No.: 16/988,306

(22) Filed: Aug. 7, 2020

Related U.S. Application Data
(60) Provisional application No. 62/884,875, filed on Aug. 9, 2019.

(51) Int. Cl. G16H 20/10; G16H 10/20; G16H 20/90; G16H 10/60; G16H 50/70; G16H 40/67; G06F 9/451; G09B 19/00; G06F 3/0482; G08B 21/24

(52) U.S. Cl. CPC G16H 20/10; G16H 10/20; G16H 20/90; G16H 10/60; G08B 21/24; G16H 40/67; G06F 9/451; G09B 19/00; G06F 3/0482; G16H 50/70

(57) ABSTRACT

A method includes receiving user-provided input(s) that include indication(s) that a user is exhibiting symptom(s) of an illness, establishing an advisory process preconfigured to assist the user in treating, using specific medications, symptoms of the illness by providing electronic reminders according to predefined time interval(s), where each reminder indicates that the user is to take respective medication(s) to treat the symptom(s) of the illness. Electronic reminders are provided according to the predefined time interval(s), and acknowledgment(s) that the user has taken at least one medication to treat the symptom(s) are tracked. Further, a kit to facilitate the method includes medications configured for treating respective symptom(s) of an illness, each medication being separately-dosed, the medications including a homeopathic medication, an antihistamine, a decongestant, and a pain reliever. The kit includes an instructional insert that includes a treatment strategy that includes a multi-day dosage regimen for treating symptoms of the illness.

### CROSS REFERENCE TO RELATED APPLICATION

This application claims priority to U.S. Provisional Application Ser. No. 62/884,875, filed on Aug. 9, 2019 entitled “PRODUCT AND APP-GUIDED COMMON COLD TRACKING AND TREATMENT”, the entire disclosure of which is hereby incorporated herein by reference.

### BACKGROUND

The common cold is one of the most frequently-occurring infectious diseases in humans. In particular, the common cold is typically transmitted via airborne droplets and is easily spread by people in close proximity. The common cold refers to a group of symptoms caused by different viruses that affect the upper respiratory tract, and is often associated with symptoms that include coughing, sore throat, runny nose, sneezing, headache, and fever. These symptoms are believed to be caused by the human body's immune response to the viral infection.

### SUMMARY

Individuals experiencing cold symptoms often take medications to provide symptomatic relief. However, due to the quantity of medication options for treating the common cold, consumers may be confused by what medication to take, dosage amounts, administration frequency, etc. Further, many consumers often attempt to self-medicate to treat symptoms of the common cold, but often these consumers are uninformed or may make incorrect assumptions. Some medications may be directed to multiple-symptom relief, in an attempt to alleviate almost any symptom associated with the common cold, but such medications may include unnecessary components that could expose consumers to potentially adverse effects without providing any benefit to the consumer. Aspects described herein can address these and other drawbacks to existing treatments for the common cold.

Shortcomings of the prior art are overcome and additional advantages are provided through the provision of a computer-implemented method. The method includes receiving, via a user interface of a user device, one or more user-provided inputs, the one or more user-provided inputs including at least one indication that a user is exhibiting one or more symptoms of an illness. Based on at least some of the received user-provided inputs, an advisory process preconfigured to assist the user in treating, using a plurality of specific medications, symptoms of the illness by, at least in part, providing electronic reminders to the user according to one or more predefined time intervals, each reminder of the electronic reminders indicating that the user is to take a respective one or more specific medications, of the plurality of specific medications, to treat the one or more symptoms of the illness. The method further provides to the user, based on the established advisory process, a plurality of electronic reminders to take the respective one or more specific medications to treat the one or more symptoms of the illness and being exhibited by the user, the plurality of electronic reminders being provided according to the one or more predefined time intervals. The method further tracks one or more acknowledgments, received from the user, that the user has taken at least one of the one or more specific medications, indicated by one or more of the plurality of electronic reminders, to treat the one or more symptoms of the illness.

Further, a kit for treating symptoms of an illness is provided. The kit includes a plurality of medications, each medication of the plurality of medications configured for treating a respective one or more symptoms of the symptoms of the illness, and each medication of the plurality of medications being separately-dosed. The plurality of medications includes a homeopathic medication, an antihistamine, a decongestant, and a pain reliever. Further, the kit includes an instructional insert that includes a treatment strategy for treating the symptoms of the illness. The treatment strategy provides a multi-day dosage regimen indicating when to individually take each medication of the plurality of medications.

Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an example cold symptom alleviation strategy, in accordance with an aspect of the present disclosure;

FIGS. 2A-2B depict an example home screen interface of an example software application, in accordance with an aspect of the present disclosure;

FIG. 3 depicts an example My Symptoms interface for receiving user inputs of daily symptoms to be tracked, in accordance with an aspect of the present disclosure;

FIG. 4 depicts an example Warning Signs interface for receiving user inputs of warning signs, in accordance with an aspect of the present disclosure;

FIGS. 5A-5B depict example My Notifications interfaces, in accordance with an aspect of the present disclosure;

FIG. 5C depicts an example schedule display for selecting a time for one or more reminder notifications to be saved, in accordance with an aspect of the present disclosure;

FIGS. 6A-6D depict example medication reminder interfaces to remind a user to take medications based on the cold symptom alleviation strategy for the user, in accordance with an aspect of the present disclosure;

FIG. 7A depicts an example My Data interface enabling a user to select user data to be copied or otherwise captured to email or share with others, in accordance with an aspect of the present disclosure;

FIG. 7B depicts an example My Data interface in which recorded user data, including medication data, corresponding to a selected date is shown such that the displayed information may be copied or otherwise captured to email or share with others, in accordance with an aspect of the present disclosure;

FIG. 7C depicts an example My Data interface in which recorded user data, including medication data, corresponding to selected dates are shown such that the displayed information corresponding to each selected date may be copied or otherwise captured to email or share with others, in accordance with an aspect of the present disclosure;

FIG. 8A depicts an example care reminder interface for providing helpful tips to a user, in accordance with an aspect of the present disclosure;

FIG. 8B depicts an example care reminder interface for prompting a user to purchase a cold symptom treatment kit, in accordance with an aspect of the present disclosure;

FIG. 8C depicts an example care reminder interface with a congratulatory notification to be provided to a user upon completion of a cold symptom alleviation strategy, in accordance with an aspect of the present disclosure;

FIG. 9 depicts an example computer-implemented method for establishing a cold symptom alleviation strategy, in accordance with an aspect of the present disclosure; and

FIG. 10 depicts a computer system in communication with external device(s), in accordance with an aspect of the present disclosure.

### DETAILED DESCRIPTION

Aspects described herein relate generally to products, processes, software, and approaches for treating a health affliction. More specifically, but not exclusively, these aspects relate to treatment kits and corresponding computer-implemented methods for treatment scheduling using medications of the kits to treat symptoms of the common cold (sometimes shortened herein to “cold”). An example computer software application/program to incorporate and use aspects described herein is the Cold FAid® mobile application offered by COLD FAid, LLC, New York, U.S.A, of which COLD FAD is a registered trademark.

In some embodiments, a cold symptom treatment kit (also referred to herein as a “kit” for short) is provided that includes medications used in conjunction with a cold symptom alleviation strategy (also referred to herein as a “strategy” or “advisory process”) to treat symptoms of the common cold, which can be particularly useful in highly susceptible populations such as, for example, college students, military cadets, frequent business travelers, healthcare providers, and teachers. This kit can reduce severity and duration of cold symptoms. In some examples, the kit may include four over-the-counter (OTC), single-active-ingredient medications, and a strategy may include a specific, easy-to-read schedule, provided as an instructional insert, for administering the medications of the kit. According to one embodiment, the strategy may include an approximately 168-hour (e.g., 7-8 days) strategy regimen for treating symptoms of the common cold. The strategy may be individualized to the needs of the individual. For example, after using the standard first 72-hour (e.g. 3-days) strategy regimen, the strategy may advocate discontinuing one or more of the medications of the kit if symptoms that a particular medication is treating have cleared. In other scenarios, the strategy may advocate for continued administration of one or more of the medications of the kit based on feedback provided by an individual for up to the remaining 96 hours (e.g. 4 days) of the strategy regimen.

According to one embodiment, the kit may include a wallet-size, easy-to-store first aid container (e.g. a red or blue zippered case, a bag, or a container that includes a first aid symbol) for convenience. This container for the kit may, according to one embodiment, be branded/marked with a QR code that an individual may scan or otherwise capture (e.g. with a portable electronic device) to provide that individual with instant access to a downloadable companion software application that assists a user in implementing and using a strategy. According to one embodiment, the downloadable software application may be provided as a “mobile app” for installation and use on the user's mobile device. In this regard, the software application may assist in scheduling or otherwise administering medications of the kit and may include engaging features for a user to interact with the software application. Example displays, interfaces, features, or other aspects for engaging the user may include but are not limited to:

MY NOTIFICATIONS: providing reminders for when to take medications;

MY SYMPTOMS: identifying and recording daily symptoms;

WARNING SIGNS: facilitating inputting information that may assist in early detection of concerning symptoms;

CARE REMINDERS: providing helpful tips during a cold;

STRATEGY TRACKER: displaying progression along the strategy schedule/regimen;

GPS: guide to locate nearby pharmacies and student health/urgent care centers;

MY DATA: log of symptom and medication information for sharing (via email, for example) with medical professionals, family member(s), and/or anyone else.

Existing approaches for treating cold symptoms have drawbacks:

CONSUMER CONFUSION: The options for treatment of the common cold seem endless when walking into a drug store looking for guidance. This is especially true for college students that look for a useful remedy in a convenient pack when they are away from home. Commonly, a parent has been the consumer advocate for the college student, rather than the student him/herself. This lack of familiarity with purchasing such items heightens the sense of perplexity.

UNINFORMED CONSUMERS AND UNINFORMED DECISION-MAKING: There is an increase in self-medication/do-it-yourself (DIY) with a wide acceptance of OTC medications by the public, media, and by medical providers. However, according to behavioral research in reference to buying OTC cold relief medications, fewer than half of customers consult a pharmacist. Commonly, marketing/media dictates, to a large extent, consumer decision-making for cold relief. Since cold sufferers often do not obtain consultation from a pharmacist, a customer can make incorrect assumptions. For example, a customer may assume that since one type of antihistamine is effective for allergy-type symptoms with a cold, that all antihistamines are effective. In fact, only first-generation antihistamines prove to be effective, and no second-generation antihistamines are effective. However, the usefulness of even the first-generation is short-lived for only the first 2 days of a cold. With this example, it is easy to see how a cold sufferer could mistakenly use the wrong cold relief products and/or use the product purchased at the wrong time.

MULTI-SYMPTOM MEDICATION: To address this frustration, the pharmaceutical industry is now trending to market combination products. These address multi-symptom relief specific to colds. With multi-symptom medication, there can commonly be an unnecessary component—medication not needed during a portion or the complete cold episode. Unfortunately, the user then takes medication that is not serving a purpose. Nonetheless, the user is still exposed to the medication's potential side effects or adverse effects.

BUYING & CONSUMER FRUSTRATION: In order to find cold relief, individuals seek out OTC or behind-the-counter (BTC) options at physical drug stores or OTC options online. Options are accessible and affordable. However, congested pharmaceutical shelves and consumer confusion creates frustration for the end-user.

POOR TIMING: The majority of buying is at the time of cold or flu symptoms. This is the time that the consumer should be at home, resting and not exposing others.

Aspects described herein may address these and other drawbacks by providing, as example, a product and strategy, and related software application for facilitating use of the product and strategy. An example kit and its companion mobile application may offer a roadmap to a plan of treatment that is supported by sound research. Advantageously, aspects disclosed herein may provide an affordable, readily available, all-in-one kit and administration protocol to treat symptoms of the common cold. This kit and associated strategy can be provided to individuals, such as individuals who are highly-susceptible to be exposed to the common cold because they interact in close proximity to other individuals (e.g. college students, frequent travelers, military personnel living in dormitories/barracks, healthcare providers, teachers, etc.). Aspects described herein may, according to one embodiment, advocate for early treatment to start the strategy at the first sign of a cold and advocate for consistent use of the medication, which is believed to alleviate the severity and duration of cold symptoms and lead to a drop in incidence of various complications. Further, the strategy may be facilitated by the software application (i.e., app), which provides a user with an automated advisory process that tracks and analyzes the user's symptoms and provides medication recommendations and notifications from onset of the common cold (strategy activation) through completion/recovery (strategy deactivation).

In particular, use of the kit and implementation of the strategy may provide a 3-tiered advantage: (1) a clear, concise strategy, (2) use of single-active-ingredient regimens combined only when needed throughout the evolution of a cold, and (3) “all-in-one” convenience. The strategy includes administration of a selection of medications, zero or more of which may be long-acting medications, to minimize frequency of medication administration. Each single-active-ingredient medication can be linked to multiple (e.g. two) identified symptoms. The strategy may be customized for the individual based on the symptoms being exhibited and the severity of those symptoms. Often, the strategy includes a standard first phase for the first 72-hours (e.g. first-3-days) of medication administration. A user may be prompted to provide feedback of symptoms at various periodic intervals (e.g. preset intervals or intervals determined at least in part by the user). Then, once specific symptoms have cleared, the medication treating that symptom may be discontinued, in accordance with aspects described herein.

Notifications or other prompts can be displayed by the software application at various points during the automated advisory process (e.g. at strategy deactivation) to encourage the user to prospectively order or buy the kit in preparation for future occurrences of the common cold. Advantageously, encouraging users to prospectively order/purchase the kit will enable users to easily administer the medications and implement a cold symptom alleviation strategy as described herein as soon as the first symptom of a cold appears, thereby maximizing effectiveness of treatment. This favors a ready-for-use alternative. Having a kit on hand for when the first symptom of a cold appears is a simple way to provide a user with peace of mind. Further, the kit may encourage consumer education with easy-to-read literature, tips, simple infographics, and a listing of references regarding the common cold.

Aspects disclosed herein may provide an effective, all-in-one cold relief product to susceptible populations in order to, for example, (1) restore health as quickly as possible with minimal symptoms and (2) incentivize self-help and responsibility with health maintenance.

According to some embodiments, the standard kit may include various modifications such as, for example, modifications specific to individuals with chronic medical concerns or other conditions. For example, the kit may include ketone testing strips for diabetics, a home serial PEF (Peak Expiratory Flow) meter/diary for asthmatics, or other customizable medication modifications. Further, to optimize sleep (e.g. for business travelers), which is critically important in alleviating cold symptoms and overcome the infection, the kit may also include ear plugs and a travel eye cover. Further, to optimize viral containment, the kit may include hand sanitizer and/or a face mask. Various other additions or modifications may be made to the kit contents.

Certain populations, such as college-age students living in dormitories as well as military personnel/cadets living in barracks often succumb to more colds than the average adult due to many risk factors: close proximity to each other, seasonal incidence of the common cold (September to April), and lifestyle. Further, those most susceptible to the common cold typically exhibit certain lifestyle habits such as, for example, sleep deprivation, emotional stress, and poor eating habits. These risk factors may impair the body's immune system, thereby rending an individual exposed to similar risk factors more susceptible to the common cold. In particular, college students with chronic medical concerns such as diabetes, asthma, and hearing loss could significantly benefit from the kit and app. For instance, this subpopulation of college students, may exhibit more adverse side effects or otherwise experience “chaos” due to the common cold.

International college students may be particularly challenged with medication selection when having a cold due to language barriers or other factors. According to one embodiment, the kit may include a package insert with the cold symptom alleviation strategy described in different languages, or the app may include a language option for selecting a language translation in which to present the strategy, including the text presented by the app to the user. Such translations may help a user better understand the strategy and ensure proper use of the medications.

Adult airline travelers also have increased susceptibility to the common cold and could benefit from a relatively small (wallet-size, in some embodiments) travel kit of medications. Similarly, those with higher-risk occupations such as healthcare professionals and teachers are also subject to greater exposure in the workplace.

Other advantages may also be realized by the app. For instance, data derived from the app could prove to be valuable. In particular, the data may be used to identify when a strategy is activated/deactivated, or the data could be used to track all stages of the cold from the beginning to recovery, as examples. The data derived from the app could be user data from one or many users, with any appropriate security measures (encryption, etc.) and/or anonymization/sanitation (removal of personally identifying information, etc.) being applied as desired. In some examples, user data collected from many users could be aggregated and analyzed. Analysis of this data can help to identify whether strategy is shortening the duration of a cold or if modifications need to be made. Additionally, this data may be used to determine a recovery duration and be used to optimize return to daily activities (e.g., help students return to class). Further, data derived from the app may provide an additional marker for determining how frequently the common cold is contracted among certain populations or in certain environments.

As noted, aspects described herein may be provided by way of a kit. Example kit contents include: medications for treating symptoms of the common cold, an instructional insert that includes a treatment strategy for treating symptoms of the common cold, a container for storing the medications, information regarding research-based studies related to effectiveness of the kit, and/or infographics for how the strategy and kit may be used together may treat symptoms of the common cold.

As indicated, the kit may include a container or other packaging that includes one or more medications. Further, an example kit may include one “homeopathic” medication and three OTC medications. In a particular example, the kit includes:
Homeopathic Medication: lozenges having a zinc compound (i.e. zinc lozenges).

Zincum Gluconicum lozenges, as one example, are registered with the U.S. Food and Drug Administration (FDA) under the category of “homeopathic” medications. As a homeopathic drug, they are registered with, but not approved by, the FDA. They are classified as a drug under the Food, Drug and Cosmetic Act. They must comply with FDA labeling requirements (21 CFR 201) and with current Good Manufacturing Practices (GMP) (21 CFR 210 and 211).
Three OTC medications: Antihistamine, Decongestant, and Pain Reliever.

For OTC medications, the FDA Center for Drug Evaluation and Research (CDER) Office of Nonprescription Drugs outlines regulations for: (1) Structured Product Labeling (SPL) and Establishment Registration (registering to pack, label and manufacture the kit), (2) obtaining an NDC (National Drug Code), and (3) labeling of OTC medications (21 CFR 201). The Code of Federal Regulations (CFR) specifies the general and permanent rules as published in the Federal Register, and chronicle monographs for over-the-counter medications that are generally recognized as safe and effective. “Monographs” define ingredients/composition for an OTC medication. Products conforming to this monograph are eligible for market with no further FDA clearance. Of the three OTC medications used in example kits, two OTC medications are considered eligible as such with monographs.

The three OTC medications used in this embodiment and their respective cited CFR listings are as follows:

(1) Antihistamine, Chlorpheniramine Maleate with monograph 21 CFR 3431.12;

(2) Nasal Decongestant, Phenylephrine HCl with monograph 21 CFR 341.20
(a); and

(3) Analgesic-Antipyretic, Naproxen Sodium with ANDA (Abbreviated New Drug Approval) 974661, labeled according to labeling monograph 21 CFR 341.85. (According to the FDA, ANDA contains data which is submitted to the FDA for the review and approval of a generic drug product. Once approved, an applicant may manufacture and market the generic drug product to provide a safe, effective, lower cost alternative to the brand-name drug it references.).

The monograph 21 CFR 341.40 “Cold, Cough, Allergy, Bronchodilator and Antiasthmatic Drug Products for Over-the-Counter Human Use” specifies the generally recognized as safe and effective use of the above noted antihistamine, decongestant and analgesic in combination. It states in subsection (c): any single antihistamine active ingredient identified in 341.12 (of which Chlorpheniramine Maleate is listed) may be combined with any single oral nasal decongestant active ingredient identified in 341.20 (a) (of which Phenylephrine HCl is listed) and any generally recognized safe and effective single analgesic-antipyretic active ingredients, or any combination of acetaminophen with other analgesic-antipyretic active ingredients, or any aspirin and antacid combination provided that the product is labeled according to 21 CFR 341.85. (The analgesic-antipyretic Naproxen Sodium qualifies within these criteria.)

Public law 109-462 of the Dietary Supplement and Nonprescription Drug Consumer Protection Act requires OTC medications to be marketed via approved FDA applications under section 505 of the FD&C (21USC 355). Labeling includes specific outlined information (title, active and inactive ingredients, purpose(s), use(s), warning(s), directions, other information, contact info for questions/reporting adverse effects). “Keep out of the reach of children” and tamper-resistance are also necessary to indicate if applicable. In accordance with such requirements, each medication included in the kit would include drug facts that are visible for a user to read (e.g. visible for review through clear plastic packaging on each box or bottle of medication). The U.S. Consumer Product Safety Commission (CPSC) authorizes child resistant closures found with many OTC products. For OTC medications used, packaging is tamper-resistant, and all boxes are left sealed in their original intact form.

Any drug combination included in the kit would be selected based on the FDA's Center for Drug Evaluation and Research (CDER), which is the agency responsible for the oversight of over-the-counter medications, validating the drugs as being effective to treat the common cold. For instance, the CDER has a published monograph in the Federal Registry (21 CFR 341.40) validating the combination of drugs described above as being generally recognized as safe and effective, as including permitted combinations of active ingredients. Further, an example embodiment of a kit has been assigned a National Drug Code number (NDC 72996-002-01) and is listed in the National Drug Code Directory available from the U.S. Food and Drug Administration.

For instance, an example kit includes a quantity of each medication to resolve symptoms of a cold (i.e. illness) within 168 hours (7-8 days depending on the time of day the treatment is started). In particular, the kit includes the following four medications:
(1) Eighteen doses of a homeopathic medication that includes a zinc compound having free ionized zinc (e.g., zincum gluconicum lozenges, at, e.g., approximately 13.3 mg per dose (1 lozenge), or zinc acetate lozenges). This number of doses (where the total amount of homeopathic medication in the kit totals at least 225 mg) enables an individual implementing a cold symptom alleviation strategy as described herein to take six doses per 24-hour period (i.e., approximately every 2-4 hours the individual is awake totaling 75-100 mg/day) for the first 72 hours (e.g. three days) of the strategy, and then discontinue use at the conclusion of the 72 hours (e.g. after the third day of the strategy).
(2) Ten doses of a first-generation antihistamine (e.g., 4 mg/pill blister pack of Chlorpheniramine Maleate or other so-called “first-generation” antihistamine). The total dosage to be taken in the first 48 hours is 40 mg, which enables an individual implementing the strategy to take five tablets per 24-hour period (i.e., approximately every 4-6 hours) for the first 48 hours (e.g. two days) of the strategy, and then discontinue use at the conclusion of the 48 hours (e.g. after the second day of the strategy). According to one embodiment, the total amount of antihistamine included in the kit is at least 40 mg. According to another embodiment, the total amount of antihistamine included in the kit is between 30 mg and 50 mg.
(3) At least thirty-five doses of decongestant (e.g., 10 mg/pill blister packs of Phenylephrine HCl). This number of doses, totaling 350 mg to be taken during the 168 hours of the strategy, enables an individual to take five pills per 24-hour period for at least the first 72 hours (e.g. three days) of the strategy with the option of continuing use up to the remaining 96 hours (e.g. totaling seven days) of the strategy. According to one embodiment, the total amount of decongestant included in the kit is at least 350 mg. According to another embodiment, the total amount of decongestant in the kit is between 100 mg and 400 mg.
(4) At least fourteen doses of pain reliever non-steroidal anti-inflammatory (NSAID) (e.g., 220 mg/pill bottle of Naproxen Sodium). This number of doses, totaling 3,080 mg to be taken during the 168 hours of the strategy, enables an individual to take two tablets per 24-hour period for at least the first 72 hours (e.g. three days) of the strategy with the option of continuing use for up to the remaining 96 hours (e.g. totaling seven days) of the strategy. According to one embodiment, the total amount of pain reliever included in the kit is at least 4,080 mg. According to another embodiment, the total amount of decongestant in the kit is between 1,200 mg and 4,000 mg.

According to various embodiments, more than the example number of dosages indicated above may be included in the kit.

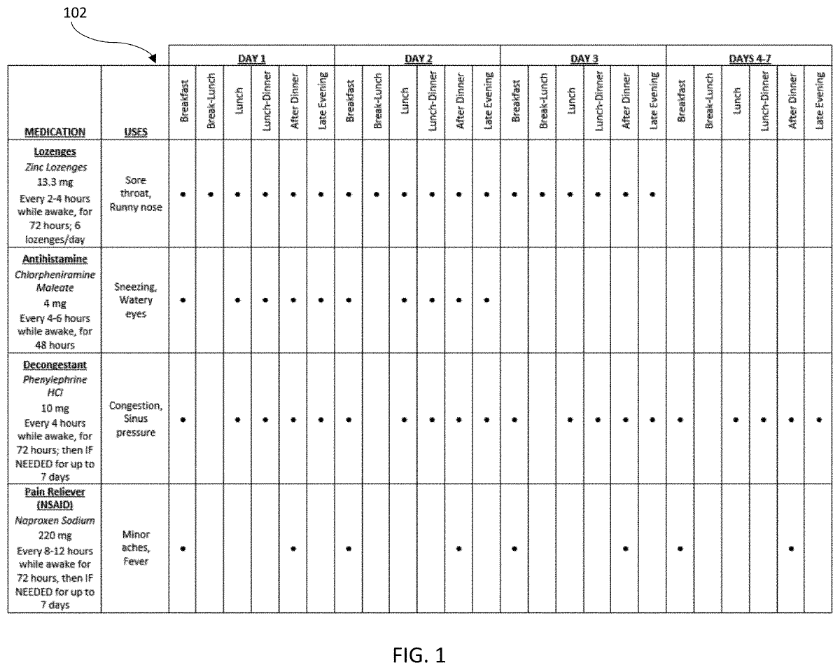

An example product insert included in the kit can include, according to one embodiment, an example cold symptom alleviation strategy chart (see FIG. 1), information about the medications or other elements included in the kit, directions for using the medications, app information about how to download the app and/or how the app may assist in implementing the strategy, possibly with example screenshots, and other informative information, such as an advisory about when to seek medical care, an advisory about when to seek consultation from a pharmacist or medical provider for additional medication(s) to address specific symptoms(s) (e.g. if there is a dry cough interrupting sleep, unrelieved congestion after 48 hours of using the decongestant in the kit, etc.), information directing the user where (online) to learn more about a provider of the kit, and a medical disclaimer. Other embodiments may include other information to inform or otherwise assist a user in implementing the strategy.

An example kit includes an instructional insert that includes a treatment strategy for treating the symptoms of the illness. The treatment strategy may provide a multi-day dosage regimen indicating when to individually take each of the medications of the kit. The instructional insert may also include a description of the kit contents, warnings, advisories, and disclaimers. For example, the instructional insert may include a description indicating:

While there is no cure for the common cold, this kit and app include the clinically-proven right medication at the right time to shorten and minimize symptoms.

An example kit warning is as follows:
IMPORTANT: Read medication information on box labels (Drug Facts, expiration dates) and this insert before using this kit.
WARNINGS: Do not take medication if allergic to any active or inactive ingredients. Ask a medical provider before use of the medications in this kit if you have a history of heart disease, high blood pressure, diabetes, thyroid disorder, stomach ulcer, breather disorder, glaucoma, difficulty urinating; or are taking sedatives or a prescription MAOI (monoamine oxidase inhibitor) drug; or are pregnant or breast-feeding or have any other complicating medical problems. Call your medical provider for advice about medication side effects. This kit is designed for ages, 18 years+; it is not for use by children (especially <12 years old). Do not exceed recommended dose of medications.

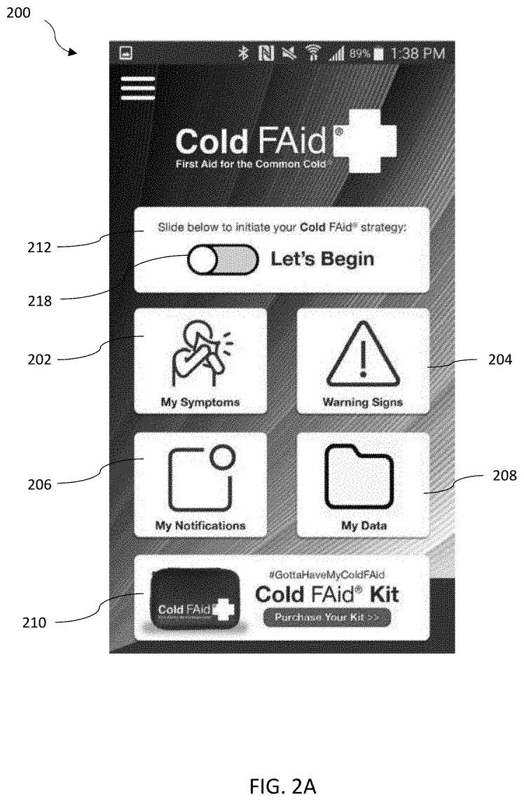

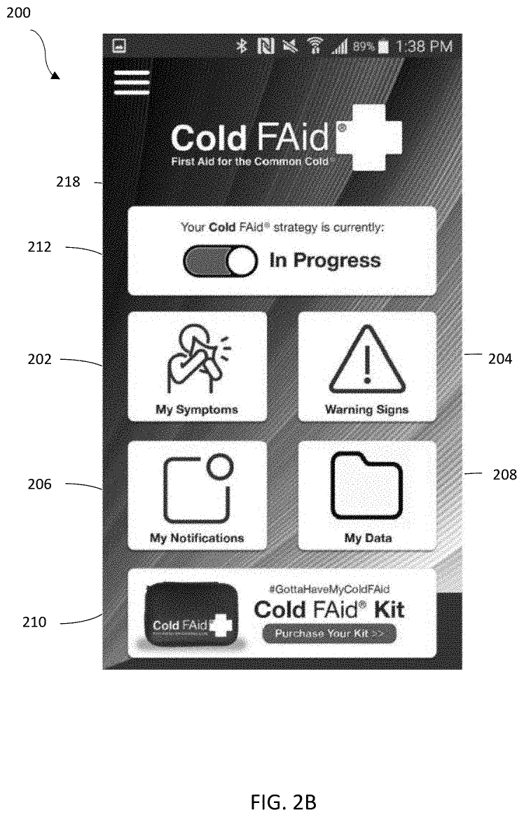

Example app information about how to download the app and/or how the app may assist in implementing a cold symptom alleviation strategy includes screenshots, such as those shown in FIGS. 2A, 3-5A, 6A-7A, and 8B, described below, informing a user how to implement an automated advisory process. The app information may include explanatory information for implementing the strategy and indicate, as examples:
MISSED DOSE NOTE: Do not take a medication if 30 minutes has passed since your last automated notification. Wait for your next notification.
NOTE: If you are traveling between time zones, do not rely on the app notifications. Use the strategy to know what to take when. Follow the medication dose frequency, not the hour of the day as this will vary according to time zone.
NOTE: Please keep in mind that email and other forms of communications over the Internet may not be secure. Unencrypted transmission, via email or other means, of privileged and/or confidential information (e.g. personal data accessed and available via the My Data module) may expose this data to interception by parties other than the intended recipient(s). The software provider accepts no responsibility for the interception or misuse of privileged and confidential information copied out of the application by the user for transmission over the Internet.

An example advisory about when to seek medical care is as follows:
Fever >101.3° F. for >24 hours, repeated shaking with chills, rash; severe headache, sever stiff neck, mental confusion, blurred vision; pain in your sinuses or face, dental pain, new loss of taste or smell; pain in your ears, loss of hearing, discharge from your ears; tonsils in your throat with a white coating, choking sensation, extreme difficulty swallowing; chest pain, difficulty breathing, coughing up blood, severe or persistent daytime cough; abdominal/back pain, repeated vomiting, inability to urinate; swollen lymph node glands in your neck, groin or armpits; extreme tiredness, weakness or muscle pain, especially if lasting >1 week; any other symptoms that seem unusual from your typical cold; and unimproved symptoms >1 week.

An example medical disclaimer indicates:
Use of this product is not intended to be used in lieu of professional medical advice, diagnosis or treatment. Always pursue the advice of a qualified medical provider with any questions you may have regarding a medical condition. Pay attention to the advice of your medical provider, and do not delay in seeking medical care due to anything you have read on the included insert, website or app. In the case of a medical emergency, call your medical provider or 911 immediately. Use of or dependence on any information provided by the provider of this product is solely at your own risk and discretion.

FIG. 1 depicts an example cold symptom alleviation strategy chart 102, which may be provided as part of the treatment strategy included in the instructional insert. The strategy chart 102 depicts a standard or default strategy for treating the common cold (i.e., an illness) that includes, according to one embodiment, a multi-day dosage regiment for taking the “homeopathic” medication and three OTC medications discussed above. The strategy chart 102 indicates, for example, a frequency (e.g. a number of hours or days) each individual medication is to be taken and indicate approximate times within each day that each medication is to be administered.

As indicated above, an example strategy may indicate:
COLD RELIEF LOZENGES: For sore throat and runny nose. Take cold relief lozenges every 2-4 hours during waking hours (i.e. 6 lozenges/day for the first three days only) starting at the earliest onset of a cold (within 24-48 hours). Suck the lozenges; do not chew. Do not swallow whole. Dissolve completely before laying down. To coat your throat, use after a meal. Do not drink or eat for 15 minutes after ingestion. Do not have citrus fruits or juices 30 minutes before or after taking as they deactivate the zinc. This kit is recommended for ages 18+. (Ages 12-17 use four lozenges per day.) Metallic taste and nausea are side effects. Power through these; the benefits of a shortened milder cold are worth it! In your kit, more tablets are provided than needed.
ANTIHISTAMINE: For sneezing and watery eyes. Take antihistamine every 4-6 hours for the first 48 hours only. Take with or without food. This antihistamine can make you drowsy. Avoid use with alcoholic beverages; both are sedating. Use caution if driving. Do not use with sedatives or tranquilizers.
DECONGESTANT: For congestion and sinus pressure. Take decongestant every 4 hours during waking hours for 72 hours then, if needed, for up to 7 days. Taking this with food may decrease stomach upset. This is a stimulant and may cause sleeplessness, nervousness, and dizziness. Stop and ask a medical provider if this occurs. Avoid excessive caffeine.
PAIN RELIEVER (NSAID): For minor aches and fever. Take pain reliever every 8-12 hours during waking hours for 72 hours then, if needed, for up to 7 days. Take this with food to protect the stomach lining. Keep well hydrated. Do not use ibuprofen (popular trade names: Advil or Motrin) when taking this. They are both the same class of medication.

Example directions associated with the strategy may indicate: For a first phase of the treatment strategy, during days 1-3 (the first 72 hours), use all medications as suggested in the example strategy. After the first phase concludes on day 3 (after the first 72 hours), if a specific symptom has cleared, discontinue use of the medication that treats this symptom during the second phase of the treatment strategy. Only continue to take the decongestant and the pain reliever if the symptoms of the illness persist and discontinue use of the decongestant and pain reliever after 7 days.

In some embodiments, included in the kit is an infographic about how the cold virus affects an individual. The infographic may include, according to one embodiment, a description that indicates: (1) how an individual contracts a virus (i.e. a virus enters a cell and multiplies; the cell bursts, thereby releasing new virus to invade nearby cells, which causes the human to exhibit the first signs of a cold such as a sore throat and runny nose); (2) how the body counterattacks the virus (i.e. the body releases inflammatory chemicals like histamines, which cause sneezing and watery eyes, and prostaglandin, which causes aches and fever); and (3) how the nose, throat, and sinuses become inflamed (i.e., swelling, drainage problems, congestion and pressure). Further, the infographic may include additional information about how a cold symptom alleviation strategy in accordance with aspects described herein may alleviate symptoms associated with the common cold. For instance, the infographic may state that the strategy uses cold relief lozenges to block viral attachment and replication, shortening the length and severity of your cold; uses an antihistamine to block histamine, reducing inflammation and deactivating your sneeze reflex; uses a decongestant to shrink swollen tissues, opening your airways and allowing drainage; and uses a pain reliever to block prostaglandin, restoring calm to muscles and normalizing your body temperature.

The infographic may also indicate: This content is not intended to be used in lieu of professional medical advice, diagnosis or treatment. If you have any questions regarding a medical condition, seek consultation with your qualified health provider.

FIGS. 2A-8C and the descriptions associated therewith depict and describe aspects of the software application (“app”) and its use. In particular, FIGS. 2A-8C depict non-limiting examples of application graphical user interfaces (GUIs; referred to herein as “interface(s)”, “display(s)” and/or “screenshot(s)”) that may be presented to a user on physical display device(s) of an electronic device. The app may include, according to various embodiments, other interactive elements or features such as, for example, a GPS feature, that are not depicted in the example interfaces of FIGS. 2A-8C.

The depicted example interfaces of the app are generally ones with which the user can interact. According to various embodiments, initially, a pop-up interface may appear requesting that the user agree with terms and conditions and a privacy policy, which the user may indicate by checking a respective checkbox. The user may have the opportunity to view the privacy policy and/or terms and conditions to which the user must agree to use the app. After the user indicates agreement with the terms and conditions and the privacy policy, the application may progress to an example ‘home screen’.

FIG. 2A depicts an example home screen interface 200 (referred to also as just “home screen” 200) of an example software application, in accordance with aspects described herein. The home screen 200 provides the user access to various components and functions of the app. One such function enables the user to initiate a cold symptom alleviation strategy (i.e. initiate processing of the app to implement and advise on the strategy for this particular user's cold) via, in this example, a prompt 212. The prompt 212 includes, in this example, a toggle 218 indicating whether a strategy is currently active. In FIG. 2A, the toggle 218 is shown in the left position with the text “Let's Begin” displayed, informing the user that a strategy is not yet in-progress. The home screen 200 also identifies a provider, its logo, and slogan (and may provide any applicable copyright, trademark, and/or patent notices). Below prompt 212, the home screen 200 includes interactive elements or modules which the user may select. In this example, such elements are My Symptoms 202, Warning Signs 204, My Notifications 206, My Data 208 links, an element 210 for ordering a kit. A brief summary of these modules on the home screen 200 are as follows:

MY SYMPTOMS 202: This module facilitates identifying and recording a user's daily symptoms via user-provided input(s);

WARNING SIGNS 204: This module facilitates receiving user input(s) of information, where the information includes warning signs that can be used to assist a user in early detection of particularly concerning symptoms;

MY NOTIFICATIONS 206: This module provides reminders of which medications to take and when (e.g., what day and dosing interval) to take the medications; and

MY DATA 208: This module provides user access to stored and recorded information, including information that was input by the user, and can be used to provide a user with a data record of, e.g., symptoms that have been logged and a record of the medications taken. The record can be made available for export, sending, saving, etc. by way of any desired means, such as via email or other electronic transmission or sharing to others (e.g., medical professionals or family).

According to various embodiments, the home screen 200 of the app may include other or additional modules or interactive elements including, for example:

STRATEGY TRACKER: An indication, such as pinpoint marker, of where or how far the user is along the strategy schedule/regimen; and

GPS: A geolocation function to assist the user in locating nearby pharmacies, medical clinics, Student Health/Urgent Care Centers, or other locations where a user may receive additional health-related assistance.

The user selects the prompt 212 (e.g. by sliding the toggle 218 to the right) to initiate a new strategy, i.e. to activate the advisory process for a cold episode. An initial tutorial display pop-up or overlay may appear over the home screen 200 or may replace the home screen 200. In any case, the tutorial display can provide any desired message and indicate activation of the app's strategy for this cold episode. As a particular example, the tutorial display provides the following message “Welcome! Let's get you feeling better! You have activated your strategy. Next, follow easy guidelines to: Log your symptoms Review warning signs, and Set up medication notifications. Additionally, according to one embodiment, a ‘Proceed to Symptoms’ interface button is presented for the user to select in order to then input symptoms. Additionally, as shown in FIG. 2B, if at any time the user returns to the home screen 200 during an in-progress strategy, after having initiated the strategy, the toggle 218 is shown toggled to the right position indicating that the strategy has been initiated and displaying “In Progress”.

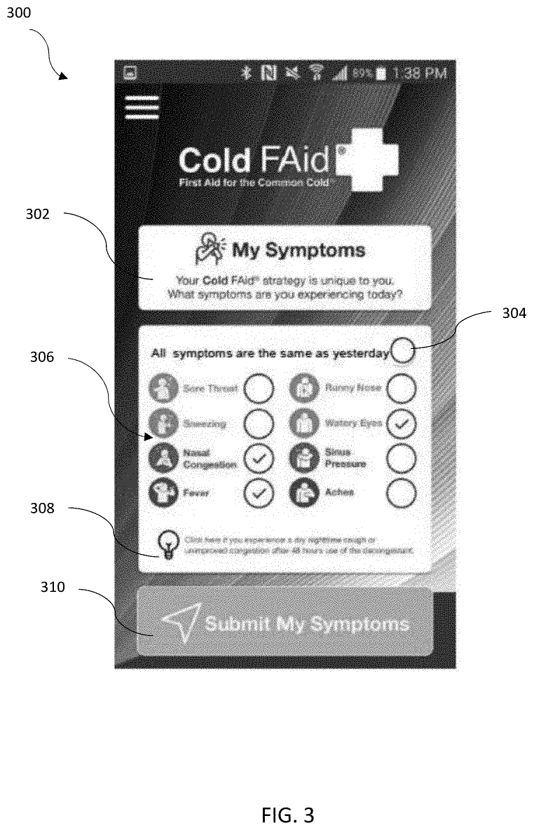

Selection of the ‘Proceed to Symptoms’ interface button or the My Symptoms module 202 may change the app display from the home screen to a My Symptoms interface. FIG. 3 depicts an example My Symptoms interface 300 for receiving user inputs of daily symptoms to be tracked, in accordance with an aspect of the present disclosure. The My Symptoms interface 300 includes a symptom prompt 302 that inquires of the user what symptoms he or she is experiencing today. The My Symptoms interface 300 also includes a plurality of selectable elements (checkboxes, buttons, etc.; “inputs”) 306. Each input of the selectable elements 306 each corresponds to a respective cold symptom. One such input 304 enables the user to indicate that the symptoms today are the same as the previous day: “All symptoms are the same as yesterday”. According to one embodiment, input 304 may not be selectable (e.g. grayed out) on the first day of the strategy (i.e. the day of activating the strategy) since at that point the app does not have record of any symptoms from the previous day. Other symptom inputs may correspond to typical symptoms that are associated with the common cold. For instance, the other symptom inputs 706 correspond in this example to a sore throat, runny nose, sneezing, watery eyes, nasal congestion, sinus pressure, fever, and aches. Additional or other symptoms may be presented. The My Symptoms interface 300 as shown includes a circular checkbox for a user to select (e.g. via touch or other input) a respective symptom, where checking the checkbox indicates presence of that symptom.

The My Symptoms interface 300 may also include an informational icon 308 that may be selected by a user. The informational icon 308 may be accompanied by text that solicits a user to select (“Click here”) the icon 308 if the user is experiencing specific symptoms listed in the text, for instance dry nighttime cough or unimproved congestion after 48 hours use of the decongestant. If the user selects the information icon, the user is presented with an informational display that includes information based on specific symptoms that the user may be experiencing. The informational display can include further advice regarding the specific symptom(s), a recommendation of additional medication to take to address the specific symptom(s), and/or a warning such as one that warns a user to discontinue use of a medication if the user is taking another medication that could cause an adverse reaction if the two medications are taken together.

Additionally, the My Symptoms interface 300 includes a submission input 310 for submitting/saving an indication of the symptoms selected (e.g. via inputs 306). The app may record what symptoms the user experienced on that given day, as indicated by the user. This input may be stored in memory and/or logged in a data log together with an indication of the date the symptom(s) were recorded.

The My Symptoms interface 300 may be automatically programmed or manually scheduled to appear (e.g. as a pop-up) periodically, such as each morning during an in-progress strategy. In this regard, the app can automatically prompt the user to indicate the symptom(s) that the user is currently experiencing.

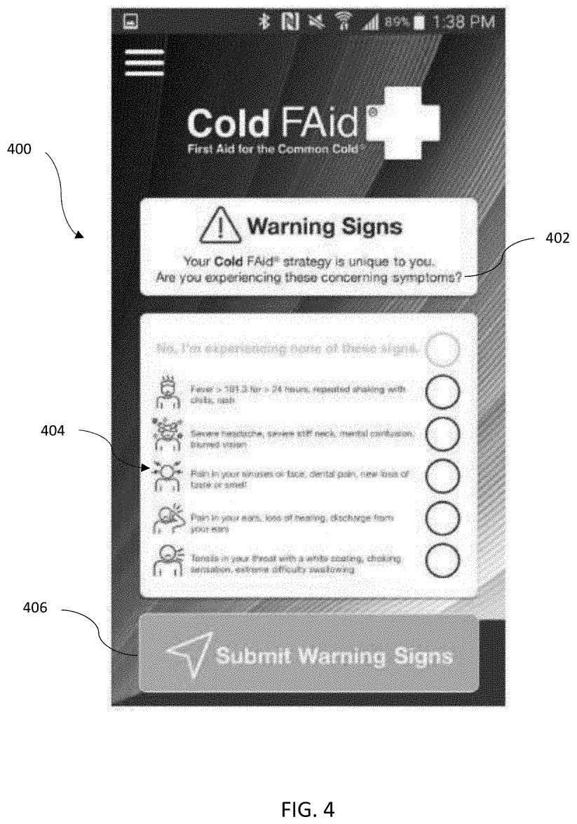

FIG. 4 depicts an example Warning Signs interface 400 for receiving user inputs of warning signs, in accordance with aspects described herein. Particular warning signs, if experienced, may trigger a notification to consult a qualified medical provider. Optionally, the Warning Signs interface 400 may be automatically presented (i) once a user has selected submission input 310 (FIG. 3) for submitting symptoms, (ii) when a user selects the Warning Signs module 204 on the home screen 200, and/or (iii) at scheduled times, for instance, each morning. The Warning Signs interface 400 interface includes a prompt 402 to a user to answer the question, “Are you experiencing these concerning symptoms?”. The user, using selection interface 404, can select via an interface element any of the presented concerning physical signs/symptoms, or select an element indicating that the user is experiencing none of the indicated signs In this example, the user clicks on the appropriate checkbox(es) to indicate presence of concerning sign(s) or symptom(s), if any. Once a user has provided an indication of whether the user is experiencing concerning physical signs or symptoms (e.g. by checking the checkbox(es)), the user may select a “Submit Warning Signs” input 406 to submit an indication of the user's selected warning signs. The app may record/log an indication of the selected concerning symptom(s), or an indication of no warning signs for this date, in memory or other storage.

According to one embodiment, an additional pop-up warning may be displayed based on the user selecting a particular concerning sign or symptom. Each concerning sign or symptom selected by the user may have an associated warning that is separately displayed. For instance, if a user selects “fever >101.3 for >24 hours” or “severe headache, severe stiff neck, mental confusion, blurred vision,” a pop-up warning may appear recommending that the user consult with a qualified medical provider. After consultation with a medical professional, the user may select and/or obtain additional medication options, such as a common medication anecdote, which the user may incorporate into the strategy. For example, if a medical professional suggests Guaifenesin ER and Pseudoephedrine HCl ER, which may not be included in the kit, the user may add these medications to the user's strategy. Based on consultation with a medical professional due to the user exhibiting one or more of the warning signs, the user may also make other medication substitutions or modifications to the strategy. For example, the user may be instructed to change dosage amounts, change the frequency of taking certain medications, discontinue use of certain medications (e.g. due to adverse side effects), etc. The strategy is not intended to be followed in lieu of following professional medical advice specific to the user.

According to one embodiment, selecting an option indicating the user is not exhibiting any of the concerning symptoms is mutually exclusive of a selection of any of the warning signs, so that if a warning sign is selected via selection interface 404, the user may not be permitted to indicate that none of the warning signs are being experienced.

According to various embodiments, the Warning Signs display 400 may be automatically programmed or manually scheduled to appear (e.g. as a pop-up) periodically, such as each morning during a an active strategy.

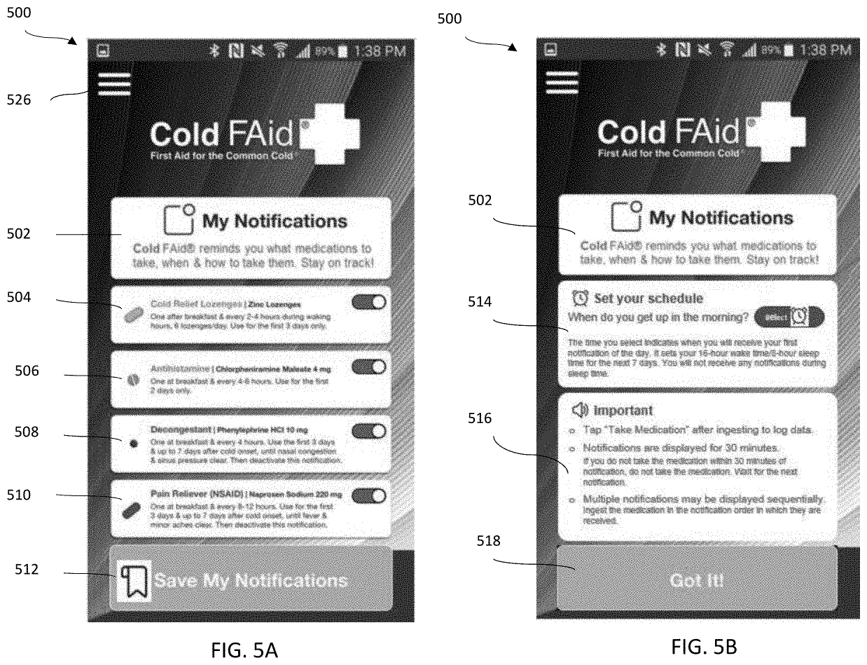

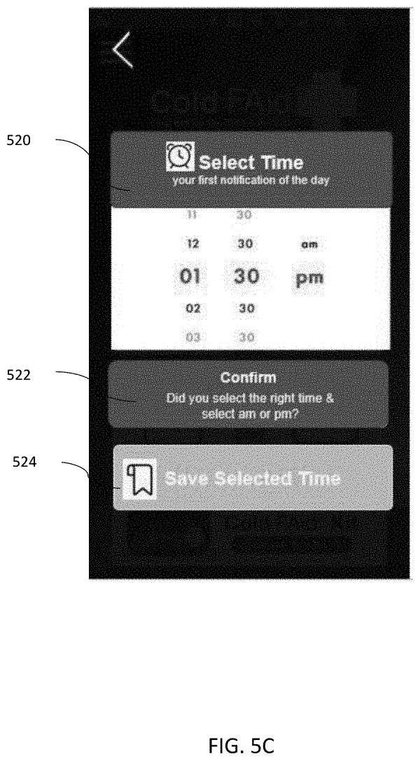

FIGS. 5A-5B depict example My Notifications interfaces, in accordance with an aspect of the present disclosure. Specifically, FIG. 5A depicts an example My Notifications interface 500 for instructing a user as to the medications included in an example kit, and when and how to take such medications. A My Notifications interface 500 may (i) be accessed when a user selects the My Notifications module 206 on home screen 200 (FIG. 2), (ii) automatically appear (e.g. at strategy activation) after a user selects the “Submit Warning Signs” input 406 on the Warning Signs display 400, or (iii) automatically after specific pop-ups with warnings have been displayed.

The My Notifications interface 500 may include an explanation 502 for how the My Notifications interface 500 may be used. The My Notifications interface 500 may allow a user to select which medications the user will take as part of the strategy or to select the medications for which the user may want notifications. The My Notifications interface may list each medication that is part of the strategy, with each medication having a toggle corresponding thereto that is selected, by default. Optionally, the corresponding medications may not be selected, and the user may individually select each medication. For instance, if the medications are not selected by default, a user may move a toggle from the left to the right for each medication for which the user wants to receive notifications, or if the medications are selected by default then the user may move the toggle from the right to the left to turn off notifications for the corresponding medication. The example My Notifications interface 500 includes Cold Relief Lozenges information 504 (with a corresponding toggle), an Antihistamine 506 (with a corresponding toggle), a Decongestant 508 (with a corresponding toggle), and a Pain Reliever 510 (with a corresponding toggle). According to one embodiment, the My Notifications interface 500 may include a toggle or other form of input element to allow a user to select all medications rather than individually selecting each medication for which the user would like to receive notifications. Once the medications for which the user desires to receive notifications are selected, the user may select a ‘Save My Notifications’ button 512.

According to one embodiment, the My Notifications interface 500 may initially include a pop-up to allow a user to select between standard notifications and custom notifications. In this regard, the user may be able to select between receiving notifications for all of the recommended medications of the strategy/regimen or receiving notifications for user-specified selected medications of the strategy/regimen as shown in FIG. 5A. Alternatively, the user may add additional medications not included in the kit as part of the strategy for which the user would like to receive notifications. According to one embodiment, standard notifications and custom notifications may be mutually exclusive options such that selection of one option will disable the other option, and the two branches (standard notifications vs. custom notifications) are straight alternatives. Alternatively, a user may select standard notifications and then select additional notifications as desired. Various other notification scheduling options are also contemplated herein.

Once a user has selected the ‘Save My Notifications’ button 512 of FIG. 5A or has otherwise indicated the medications for which the user would like to receive notifications, the My Notifications interface 500 may be modified, as shown in FIG. 5B, to allow a user to select a schedule for receiving notifications. FIG. 5B depicts an example My Notifications interface through which a user configures options to select reminder notifications to take one or more medications of the strategy. To set a schedule, a user may select a ‘Set Your Schedule’ element 514 for selecting a notification schedule based on a selected time for the first notification of the day, corresponding to a set wake-up time. The My Notifications interface 500 may be modified, as shown in FIG. 5C, to display a Select Time interface 520. The app can enable a user to select a set notification schedule for receiving notifications according to a recommended ingestion frequency, select another pre-set notification schedule, or select a completely customized notification schedule. Each notification time may be individually selected via a Select Time interface such as that of FIG. 5C. According to one embodiment, a user may be prompted, via prompt 522, to review the date/time selected to make sure the user input the correct date/time. Further, the user may save the selected time via a Save Selected Time button 524. According to one embodiment, the My Notifications interface 500 may allow a user to answer a set of questions about the user's schedule (e.g. their routine mealtimes, routine bedtime, routine daily rise time, waking hours, etc.), upon which the notification schedule for taking medications can be based. Various other scheduling parameters or notification parameters may also be customized, according to various embodiments.

Returning to FIG. 5B, the My Notifications interface 500 may also include instructions 516 indicating to a user how to record or log that the user has taken a medication, how long a notification will be displayed and what to do if the user skips taking the medication or does not otherwise take the medication according to the notification schedule, or indicate a sequence for taking the medications. A ‘Got It!’ button 518 or other button is provided to complete the notification setup and exit the My Notifications interface 500.

According to one embodiment, at any time a user may select the menu button 526 (FIG. 5A) (sometimes referred to as the ‘hamburger’ button) on the top left on the My Notifications interface 500 to change notification preferences (e.g. change what medications to take or add user-specific medications recommended to the user by a medical professional and not included in the kit).

According to one embodiment, preset notification schedules may be selected for medications not included in a kit. For example, when a user selects a particular medication, such as Guaifenesin ER or Pseudoephedrine HCl ER, a user may select a preset notification schedule. For instance, the schedule may include:

For Guaifenesin ER—The first notification to take a pill would be for 8:00 AM or 8:00 PM, whichever is closer to the time of activation of the strategy. It may be preset to continue with twice-daily notifications every 12 hours until the user deactivates the notification or until 7 days after cold onset (up to 7 days, 14 notifications).

For Pseudoephedrine HCl ER—The first notification to take a pill would be for 8:00 AM or 8:00 PM, whichever is closer to the time of activation to take that medication. It may be preset to continue with twice daily notifications every 12 hours until user deactivates the notification or for the total of 7 days after cold onset.

According to one embodiment, a user may be prevented from scheduling notifications for two medications that should not be used together. For instance, once the REGULATED decongestant (Pseudoephedrine HCl ER) is selected for the user to receive notifications, then the user would be prevented from also setting notifications for the UNREGULATED decongestant (Phenylephrine HCl).

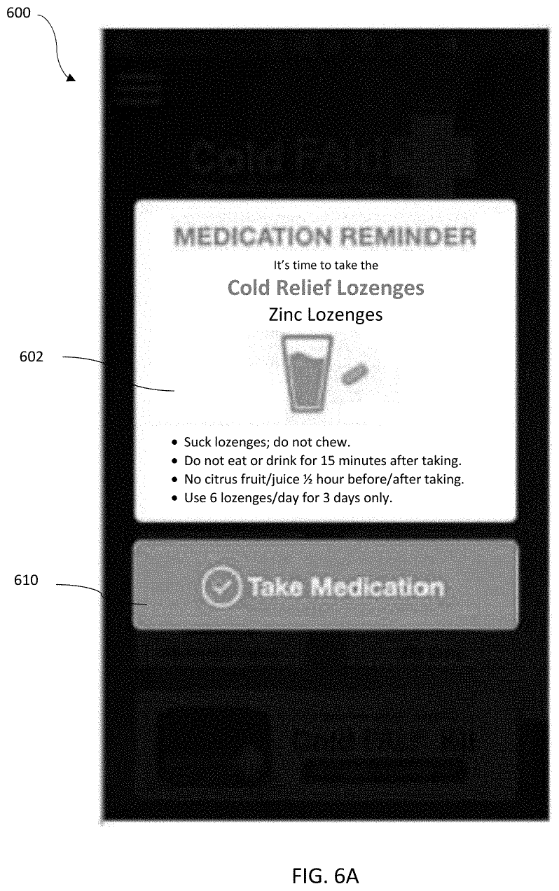

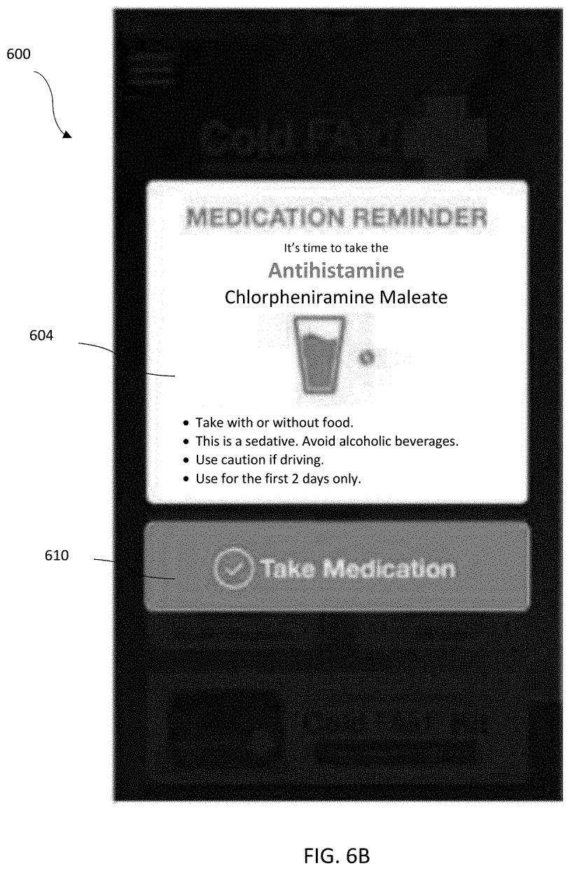

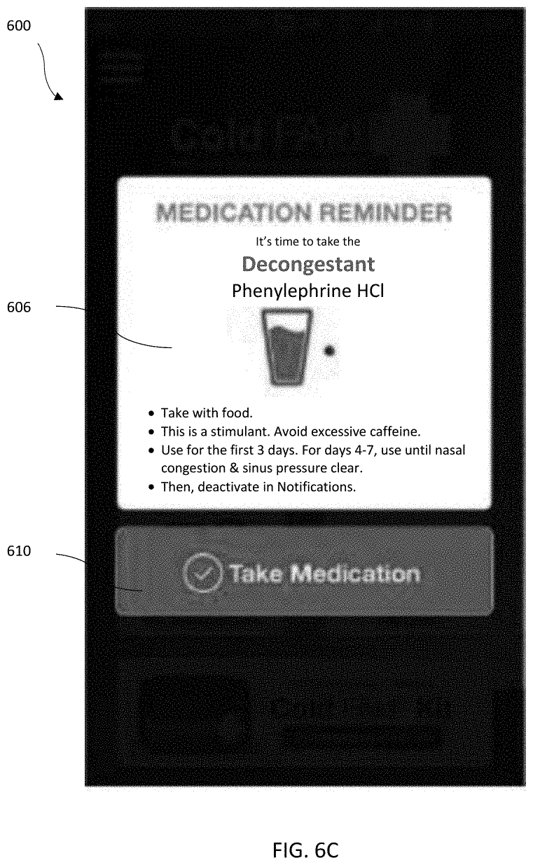

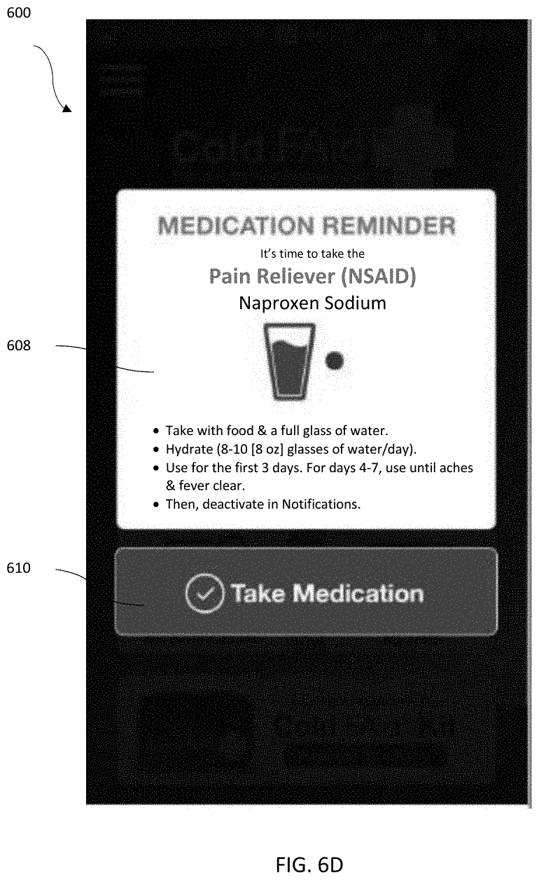

FIGS. 6A-6D depict example medication reminder interfaces 600 to remind a user to take medications based on the cold symptom alleviation strategy for the user, in accordance with an aspect of the present disclosure. In particular, a medication reminder interface may indicate to a user which medication(s) to take at a given time that the interface is presented (e.g. as a notification, pop-up, alert, or the like) and/or how to take the medication. It is noted that medication reminder interfaces 600 depicted in FIGS. 6A-6D and elsewhere show medication reminder interfaces 600 overlaying the app in the background. However, it is noted that medication reminder interfaces may be distributed, shown, provided, conveyed, or the like to users in other/additional formats via an electronic device, for instance via text, email, phone call, alarm, badge-style notification, or any other appropriate notification mechanism. The app of the user's mobile electronic device may be paired or otherwise in communication with one or more other electronic devices, for instance via an IoT environment, to notify a user via those one or more other electronic device(s) other than the user's mobile electronic device. Further, notifications may be or include a sound (e.g., a ring, a tone, an announcement, an alarm), possibly in conjunction with a visual pop-up (e.g. a banner) on a screen of the user's mobile electronic device, or other format. For instance, a notification may be provided via an OS-provided notification facility. In this manner, the app does not necessarily need to be loaded in the foreground of the mobile device's operating system execution in order for the device to serve the appropriate notifications to the user at the appropriate times.

FIG. 6A depicts an example medication reminder interface 600 directed to the Cold Relief Lozenges medication. The interface 600 of FIG. 6A shows notification 602 presented to a user as part of the strategy in accordance with the notification preferences selected by the user for Zinc Lozenges. According to one embodiment, the Cold Relief Lozenges notification 602 may be presented to the user at 8:00 AM (corresponding to wake or breakfast time) and may be presented throughout the day at select intervals based on times the user has selected, or based on recommended times for taking the Cold Relief Lozenges. For example, the Cold Relief Lozenges notification 602 may continue to be presented, at 2-4-hour intervals (depending on the interval selected by the user), throughout the day until a selected end time, such as 12:00 AM when the user is scheduled to go to bed. Various other schedules are possible depending on the notification schedule selected using the My Notifications interface 500. In a specific example, the Cold Relief Lozenges notification 602 is presented at 3-hour intervals. Additionally, the Cold Relief Lozenges notification 602 that may indicate how the user is to take the medication. For instance, the example Cold Relief Lozenges notification 602 indicates that the user is to suck the lozenge rather than chew the lozenge, that the user is to not eat or drink for 15 minutes after taking, that the user is not to have citrus fruit or juice ½ hour before or after taking the medication, and that six lozenges are to be taken for the first three days of the strategy.

FIGS. 6B-6D depict other example medication reminder interfaces 600. In particular, FIG. 6B depicts an example interface 600 with an antihistamine notification 604, FIG. 6C depicts an example interface 600 with a decongestant notification 606, and FIG. 6D depicts an example interface with a pain reliever notification 608.

The medication reminder interfaces 600 may include a ‘Take Medication’ button 610 for the user to affirmatively indicate that the user has taken the medication and/or to dismiss that notification. In order for the system to record that a medication has been taken and save the user's response, the user may be required to tap the “Take Medication” button 610. Once a user selects the “Take Medication” button 610, the system may then record what medication was taken and when the medication was taken. According for various embodiments, rather than relying selection of the “Take Medication” button 610 to record what medication was taken and when the medication was taken, a user may also separately record what medication was taken, when a medication was taken, or otherwise edit the record of medications taken (e.g., if a user accidentally selects the “Take medication” button 610 without taking the medication or if a user takes a medication outside of the time period during which the medication reminder interface 600 is being displayed). According to one embodiment, a user may “snooze” (e.g. via swiping the screen of the electronic device) or otherwise request a temporary dismissal of the notification and later reappearance after a set period of time (e.g. 5 minutes) if, for example, the user needs to continue using the screen of the electronic device and is unable to immediately take the medication. Various other notification features may also be incorporated in the app to allow a user to modify how a notification is distributed, presented, acknowledged, and responded-to.

According to one embodiment, a notification may automatically self-resolve or otherwise turn off after a period of time (e.g., 30 minutes) such as, for example, if a user does not dismiss and/or respond to the notification or otherwise indicate that the medication has been taken. For instance, notifications 602, 604, 606, and 608 may expire, and the respective interfaces 600 dismissed, after any configurable or desired timeframe. As indicated above, if a user does not take a medication within a set period of time after the notification is presented, the user may be advised to skip that dose of the medication and wait until a next notification for that medication. According to one embodiment, this warning may automatically appear on the screen if a user does not dismiss the notification or otherwise indicate that a medication has been taken.

Further, according to one embodiment, if multiple medications need to be taken at the same time, (e.g. 8:00 AM), then rather than sending four notifications at the same time, all of which the user is to individually indicate as being taken, a single medication reminder interface 600 may indicate that the user is to take the multiple medications at a single time.

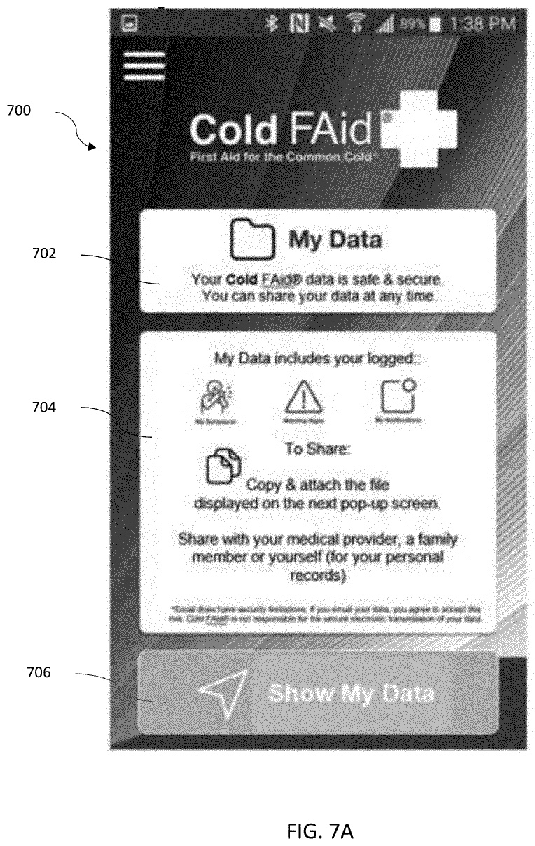

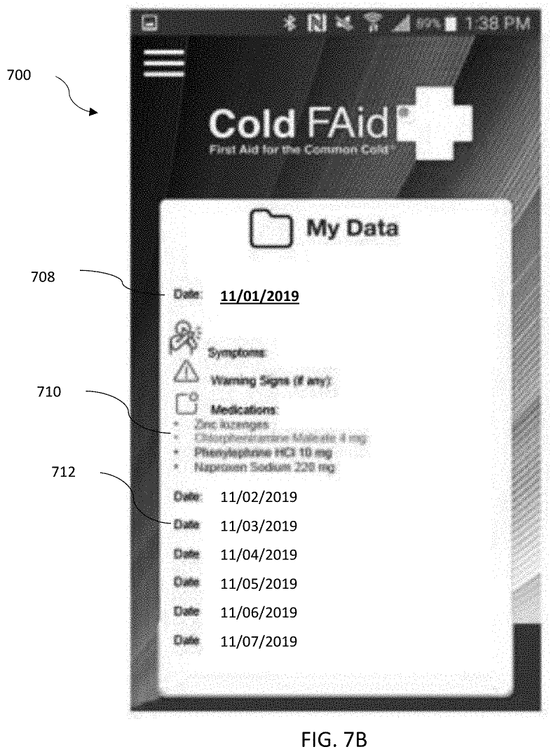

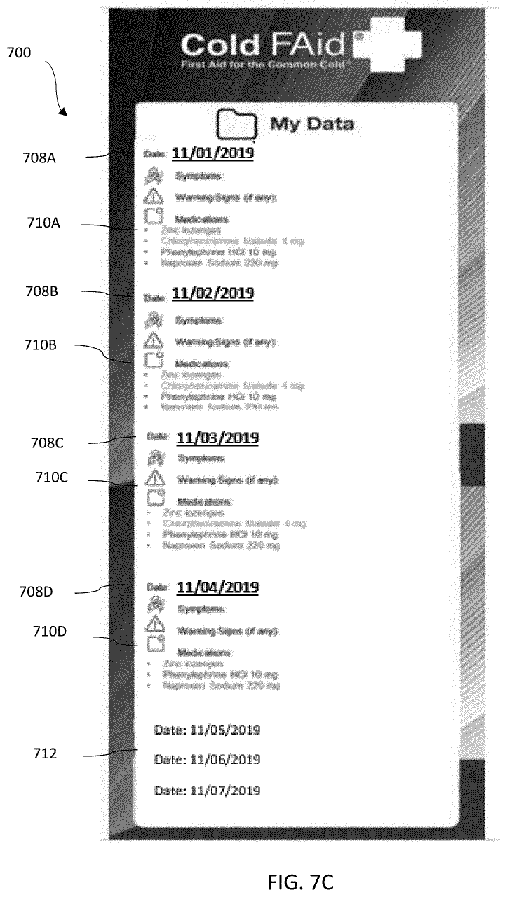

FIG. 7A depicts an example My Data interface 700, which may enable a user to select user data to be copied or otherwise captured to email or share with others. As noted previously, once a user provides information in the form of a user input, such as selections to indicate that a given amount of medication was taken at a given time, the application can record the input to a data log or other data structure in working memory (RAM) and/or other storage. The record of the information input by the user may be accessed via the My Data display 700 and stored locally on the device and/or remotely in any secured form, such as in encrypted form using a data encryption framework.

The My Data interface 700 of FIG. 7A includes an initial instructional screen with an informational block 702 indicating that the data is safely and securely stored (for instance in encrypted form on the mobile device and/or at one or more remote systems) and may be shared at any time. Further, the My Data interface 700 of FIG. 7A includes an instructional block 704 indicating a type of user information that has been recorded (e.g., via a My Symptoms icon, a Warning Signs icon, a My Notifications icon), and instructions for how to share the recorded data. A user may then select a Show My Data button 706 to access this personal data.

The My Data interface 700 of FIG. 7A may be accessed and viewed at any time by selecting the My Data module on the home screen (e.g. My Data 208 on home screen 200 of FIG. 2A).

The data, when selected for display, may be presented in any desired format, for instance a format in which the data may be easily copied for the user to share the personal information. Sharing could occur via a third-party service and/or software external to the app, such as via an email to send to the target recipient of the data. For instance, a user may select personal data displayed on an interface of the app to copy the data to the computer system's ‘clipboard’ feature or save the selected data to a file. The copied or saved data would then be attached to an email or copied into a body of an email, as examples. Alternatively, according to one embodiment, a user may take a screenshot of the interface displaying personal data and distribute the screenshot to one or more other target recipients via text, email, or other electronic communication service.

FIG. 7B depicts an example My Data interface 700 in which recorded user data, including medication data, corresponding to a selected date is shown in a format that may be copied or otherwise captured to email or share with others. The My Data interface 700 of FIG. 7B includes a list of selectable dates (e.g. consecutive dates of Nov. 1, 2019 through Nov. 7, 2019), the selection of any one of which expands in a drop-down fashion to present any recorded user data corresponding to that date. For instance, as shown, it indicates any symptoms selected and recorded on that day, any warnings issued on that day, and any medications (including dose rate) taken on that day. Alternatively, according to some embodiments, each listed date may, by default, show any recorded information associated with that date without the user having to manually select the date to view the data associated therewith. According to one embodiment, a user may select (e.g. via an electronic calendar) a date or a date range in order to access records for the desired date(s).

After date(s) have been selected, for instance by tapping on the date(s) for which recorded data is to be displayed, the dates are displayed in the My Data interface. As shown in FIG. 7B, the user has selected a first date 708 (e.g. Nov. 1, 2019) to expand the view of data associated with that date, and individual data 710 corresponding to that selected date is displayed. As an example, an indication of the three types of data are shown, i.e. Symptom data (“Symptoms”), Warning Sign data (“Warning Signs (if any)”, and Medications data (“Medications”), with corresponding icons. In this example, there is no symptom or warning sign data for Nov. 1, 2019, but there is medications data, which is listed in FIG. 7B. Other dates 712 (e.g. Nov. 19, 2002-Nov. 7, 2019) are listed for the user to select one or more if the user desires to see the corresponding data for the corresponding one or more other dates.

According to one embodiment, the My Data interface 700 for showing personal data may, by default, display labels for dates corresponding to a current (in-progress) strategy, with the a first-listed date (e.g. 708) being the first day when the current strategy was started, and the following dates 712 being the remaining dates of the current strategy. For example, the first date 708, e.g. Nov. 1, 2019, of the strategy may be the first date listed on the My Data display 700, with remaining dates, e.g. Nov. 2, 2019-Nov. 7, 2019, being the remaining dates of the strategy. It is noted that “remaining dates” could refer not only any passed dates up to a current date (that day), but optionally could list the dates in the future to which the strategy is expected to extend. For example, if the strategy is expected to last 8 days total, then the interface could list all 8 dates even if a current day is only the 4^{th} day into the strategy. In this example, there would be no recorded data for at least days 5 through 8 because they have not yet occurred.

FIG. 7C depicts an example My Data interface 700 in which recorded user data, including medication data, corresponding to selected dates are shown such that the displayed information corresponding to each selected date may be copied or otherwise captured to email or share with others, according to one embodiment. The example of FIG. 7C is very much like the example of FIG. 7B except that in FIG. 7C the user has selected dates Nov. 2, 2019 through Nov. 4, 2019 (708A-708D) for which a record is shown. Alternatively, these may have been expanded by default. In one example, the first four dates 708A-708D (i.e. Nov. 1, 2019-Nov. 4, 2019) are the only dates that have occurred and the remaining dates 712 (i.e. Nov. 5, 2019-Nov. 7, 2019) have not yet occurred, so only the dates that have occurred are automatically expanded without any user selection thereof needed. In any case, the four selected dates 708A-708D each include a corresponding record of symptoms, warning signs and medications taken (including dose rate) on each respective date. The medications taken may vary depending on the day, as shown, for instance when comparing the first date 708A (i.e. Nov. 1, 2019), in which Chlorpheniramine Maleate 4 mg was taken, with the fourth date 708D (i.e. Nov. 4, 2019), in which the Chlorpheniramine Maleate 4 mg was not taken and therefore is not shown. Further, the remaining dates 712, that are unselected and/or have not yet occurred, have no recorded information displayed thereunder.

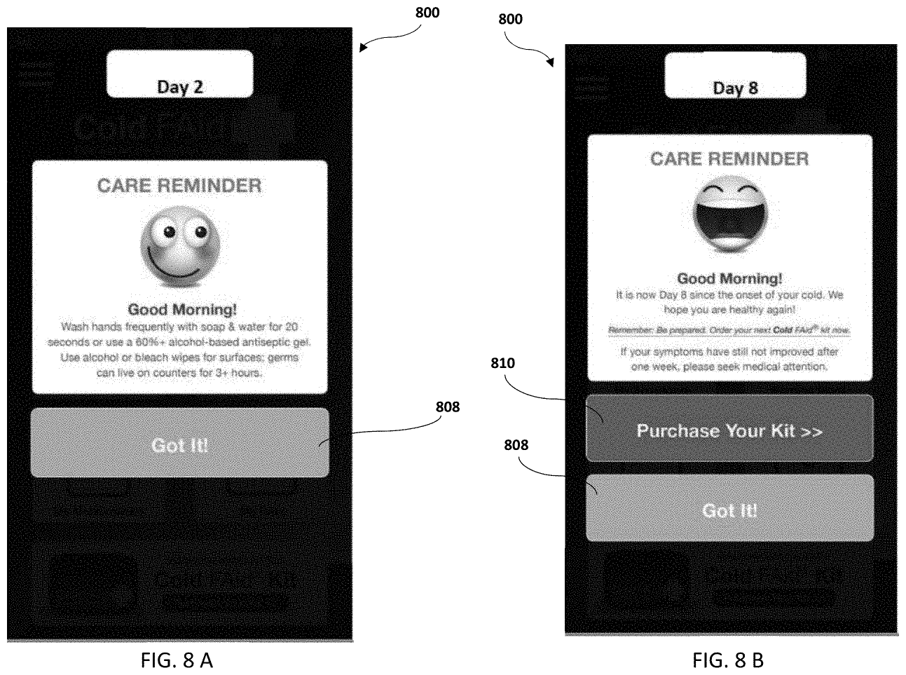

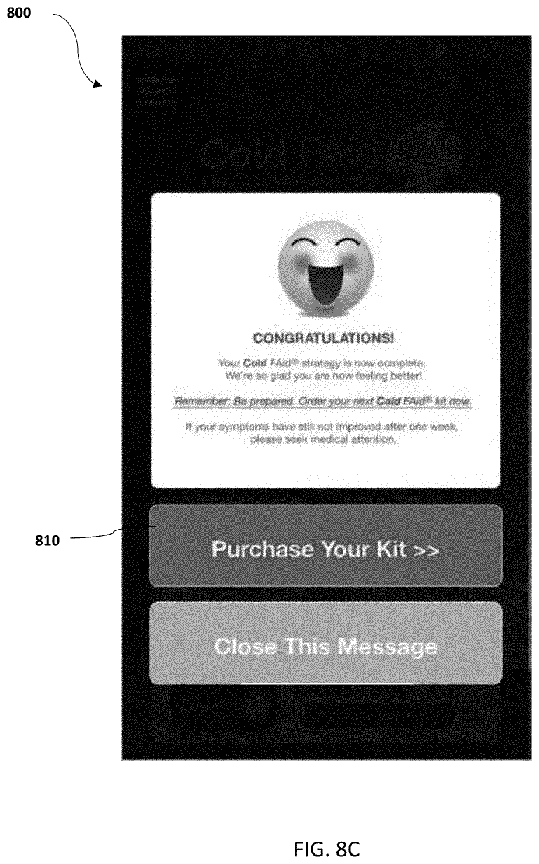

Care reminders can be selectively displayed, for instance at defined times. FIGS. 8A-8C depict example care reminder interfaces 800 for provision to the user by the app. These care reminder interfaces may be displayed at periodic intervals, such as once per day for each day of the strategy/regimen, and/or at particular times during each day (or selected days) of the strategy. For instance, FIG. 8A depicts an example care reminder interface 800 that may be provided to a user on a second day (“Day 2”) of the strategy. The care reminder can present any desired reminder(s) and/or other information for the user. The Day 2 reminder is directed generally to hand and surface sanitation. FIG. 8B depicts an example care reminder interface 800 provided to a user on an eighth day (e.g. the final day) of the strategy. According to one embodiment, a care reminder 800 may include a cheerful initial greeting each day, a useful tip when enduring the common cold, a warning, a reminder to order a new kit towards the end of the current strategy, and/or other desired unprompted communication.

According to one embodiment, each day may begin with presentation of a care reminder presented at the user's configured breakfast time (for instance 8 AM). To dismiss a care reminder interface 800 from the display screen, a user may select a button 808, swipe the screen (e.g. up, left, right), or perform any other required interaction/input. Additionally, as in the case of FIG. 8B, the care reminder interface 800 may be dismissed from the display screen if the user selects a button 810, in this case a button to link/direct the user to a website or in-app purchase screen that provides functionality enabling the user to purchase a new kit.

FIG. 8C depicts an example care reminder interface 800 with a congratulatory notification to be provided to a user upon completion of a cold symptom alleviation strategy in accordance with aspects described herein. The congratulatory notification may be provided once a user has selected the Got It! button from the care reminder of the last day of the strategy (e.g. selects 808 of FIG. 8B). The congratulatory notification of the interface 800 of FIG. 8C indicates that the user should seek medical attention if the symptoms still have not improved after completion of the strategy.

According to various embodiments, a user may also shop for products via the app by, for example, selecting an interactive element (e.g. 210 of FIG. 2A, 2B). Additionally, as depicted by care reminders presented in FIGS. 8B and 8C, the user may select a ‘Purchase Your Kit >>’ button 810, and the app can present to the user an in-app shopping interface for ordering additional product(s), including one or more kits. According to one embodiment, the in-app shopping interface may provide price, shipping information, and at least some product information. A desired website (for instance the website of the provider of the kit/product) may be referenced for additional information and/or a third-party website indicating a nearby shop or store that may have the kit in stock for the user to purchase may be referenced. In the example of an in-app shopping interface in-app shopping, the user may select an ADD TO CART button and a shopping cart interface may be displayed. Quantity selection, pricing, payment selection and the option of a personal message for the package may be presented therein. Any appropriate order processing facility/facilities can be incorporated into or integrated with the app to facilitate a purchase transaction and initiate shipment of any ordered item(s). For instance, an app payment gateway/facility and order processing facility may be connected to and/or in communication with a website, such as the website of the kit provider, via an Application Programming Interface (API) of a third-party, an example of which is Shopify. Orders can then be processed through such third-party.

In some examples, the kit may be sold via online retailer(s). Additionally, or alternatively, it can be sold to and/or via brick-and-mortar retailers, such as college campus bookstores, for direct sales to customers. According to various embodiments, the kit may be licensed or otherwise subscribed-to as part of a business-to-business-to-consumer model. For instance, the kit may be distributed as: (1) a component of a health fee assessed by an educational institution, such as a college or university, that students typically pay, with the kit being provided to students upon enrollment or on a move-in day; (2) a product available at the student health center for student sick visits, offering the kits to students who are seen early in the process of a cold.

As a specific example, demographic studies reveal that college students' influenza annual vaccination rates precipitously drop after high school. Partnering with a third-party entity may raise awareness and need for young adults or students to be prepared with a kit.

According to one embodiment, the app may include or leverage a GPS (Global Positioning System) module and/or a link to a third-party GPS facility (app, website). In a specific example, a GPS module can be viewed at any time by the user selecting a menu interface element, such as the one shown on the top left of the home screen 400 (FIG. 4). The GPS module, when invoked, may provide the ability for the user to search for locations of nearby pharmacies, student health centers, urgent care centers, or the like, if needed, and then present a list of nearest such facilities. The results could be presented in a list view or a map view (shown on a map), according to user preference. The list functionality can be selectively configured such that it lists only those establishments that are known to stock, or that are expected, likely, or presumed to stock, the kit for purchase. In other examples, the list is built based on the user's input keywords or search parameters without regard to whether the listed establishments may carry the product.

At a conclusion of the strategy, a user may deactivate (or ‘power-off’) the strategy. For instance, a user may toggle-off or deactivate all medication notifications, in which case the app may automatically deactivate the strategy. Additionally, or alternatively, the user may be able to slide the toggle 218 of the home screen 200 (see FIG. 2B) to deactivate the strategy and/or indicate to the app that the strategy for the user's recent cold episode can be deactivated.

Alternatively, the strategy may automatically deactivate after displaying the congratulatory notification of FIG. 8C (e.g. on day 8). Various other deactivation approaches are also contemplated herein.

After strategy deactivation, a future launch of the app could present the interface of FIG. 2A for a subsequent strategy activation in connection with the onset of a possible new cold episode.

Data analytics can track when the strategy is activated and when it is completed or deactivated. This can provide data on the effectiveness of the kit. In some embodiments, the data is returned to a remote entity, such as a remote computer system operated by or on behalf of the provider of the kit or an affiliate thereof, for analysis of the returned data.

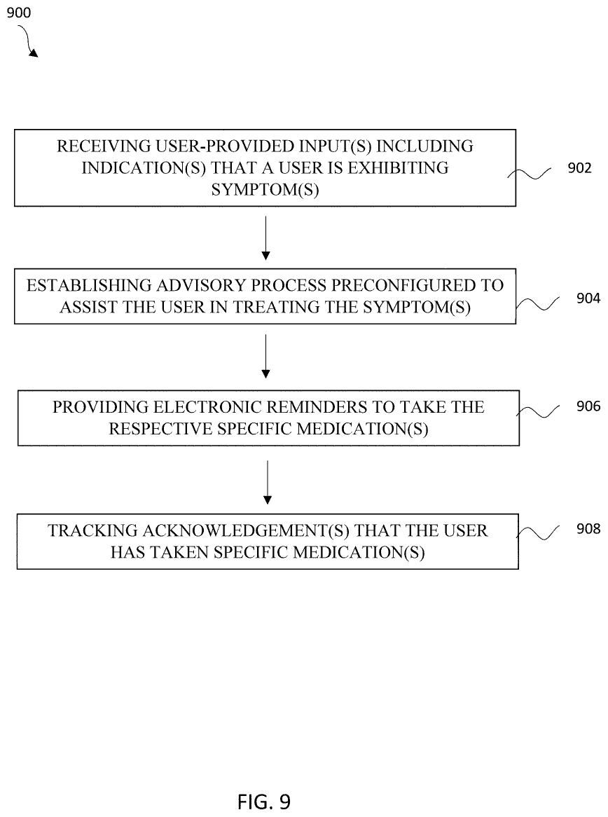

FIG. 9 depicts an example computer-implemented method 900 for establishing a cold symptom alleviation strategy in accordance with aspects described herein. The method includes receiving 902, via a user interface of a user device, one or more user-provided inputs, the one or more user-provided inputs including at least one indication that a user is exhibiting one or more symptoms of an illness. Further, based on at least some of the received user-provided inputs, the method establishes 904 an advisory process preconfigured to assist the user in treating, using a plurality of specific medications, symptoms of the illness by, at least in part, providing electronic reminders to the user according to one or more predefined time intervals, each reminder of the electronic reminders indicating that the user is to take a respective one or more specific medications of the plurality of specific medications to treat the one or more symptoms of the illness. Additionally, the method provides 906 to the user, based on the established advisory process, a plurality of electronic reminders to take the respective one or more specific medications to treat the one or more symptoms of the illness and being exhibited by the user, the plurality of electronic reminders being provided according to the one or more predefined time intervals. The method also tracks 908 one or more acknowledgments, received from the user, that the user has taken at least one of the one or more specific medications, indicated by one or more of the plurality of electronic reminders, to treat the one or more symptoms of the illness.

Further, the one or more predefined time intervals can be either (i) preconfigured as part of the advisory process, or (ii) defined by the user as an input of the one or more user-provided inputs. Additionally, the one or more predefined time intervals can (i) align with intended dose times that the user is to take the respective one or more specific medications, or (ii) align with a user-defined amount of time prior to the intended dose times that the user is to take the respective one or more specific medications.

The method may also periodically issue, during the advisory process, a prompt asking for symptom updates, the prompt may instruct the user is to indicate any symptoms of the illness that the user is still exhibiting. Additionally, the method can include receiving, from the user, a selection that indicates for which of the respective one or more specific medications, of the plurality of specific medications, the user would like to receive the plurality of electronic reminders.

Further, the preconfigured advisory process can include a timeout for automatically dismissing a reminder of the plurality of electronic reminders provided to the user based on determining that no acknowledgment, that the user has taken a dose of medication corresponding to the reminder, has been received from the user. The automatic dismissal can issue an instruction to the user to skip that dose of medication corresponding to the reminder. Also, the issued instruction can indicate to the user to resume taking the respective one or more specific medications at a next scheduled dose time.

The plurality of specific medications of the preconfigured advisory process can include at least four specific medications. For instance, the plurality of specific medications of the preconfigured advisory process can be selected from a homeopathic medication, an antihistamine, a decongestant, and a pain reliever.

The method can also include recording, as a user record, (i) at least one of the one or more user-provided inputs, the user record of the one or more user-provided inputs indicating any of the one or more symptoms of the illness the user is continuing to experience during a time during which the advisory process is being followed, and (ii) at least one of the one or more acknowledgments being tracked, the user record of the one or more acknowledgments indicating a dose history. Further, based on the recording, the user record can be provided to the user, where the user record is accessible by the user to share with one or more individuals.

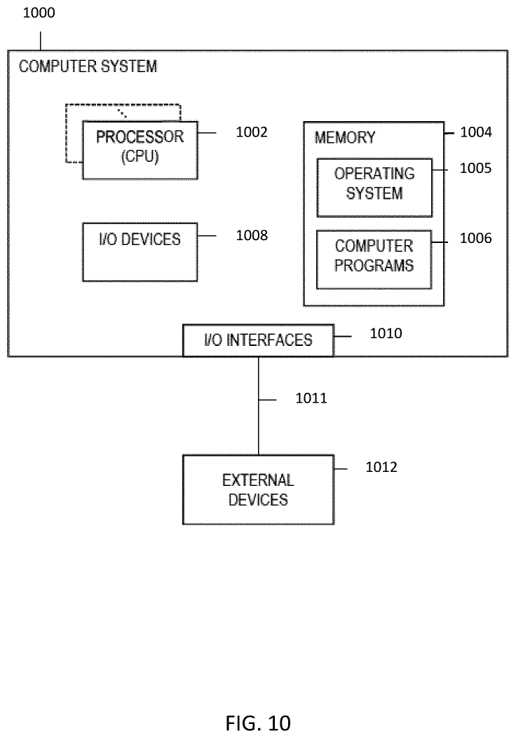

Processes described herein, such as those followed by an app as described here and presented by way of interfaces described herein, may be performed singly or collectively by one or more computer systems. In some examples, a mobile device executing the described app is performing aspects of processes described herein. The mobile device may be in communication over one or more networks with backend server computer system(s) which may be providing processing facilities for processing also described herein. In this regard, the mobile device may be running in a client-server configuration with one or more servers. FIG. 10 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, Calif., USA) or ARM Holdings plc (Cambridge, England, United Kingdom), as examples.

FIG. 11 shows a computer system 1000 in communication with external device(s) 1012. Computer system 1000 includes one or more processor(s) 1002, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1002 can also include register(s) to be used by one or more of the functional components. Computer system 1000 also includes memory 1004, input/output (I/O) devices 1008, and I/O interfaces 1010, which may be coupled to processor(s) 1002 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 1004 can be or include main or system memory (e.g. Random-Access Memory) used in the execution of program instructions, storage device(s) such as hard drive(s), flash media, or optical media as examples, and/or cache memory, as examples. Memory 1004 can include, for instance, a cache, such as a shared cache, which may be coupled to local caches (examples include L1 cache, L2 cache, etc.) of processor(s) 1002. Additionally, memory 1004 may be or include at least one computer program product having a set (e.g., at least one) of program modules, instructions, code or the like that is/are configured to carry out functions of embodiments described herein when executed by one or more processors.

Memory 1004 can store an operating system 1005 and other computer programs 1006, such as one or more computer programs/applications that execute to perform aspects described herein. Specifically, programs/applications can include computer readable program instructions that may be configured to carry out functions of embodiments of aspects described herein.

Examples of I/O devices 1008 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (1012) coupled to the computer system through one or more I/O interfaces 1010.

Computer system 1000 may communicate with one or more external devices 1012 via one or more I/O interfaces 1010. Example external devices include a keyboard, a pointing device, a display, and/or any other devices that enable a user to interact with computer system 1000. Other example external devices include any device that enables computer system 1000 to communicate with one or more other computing systems or peripheral devices such as a printer. A network interface/adapter is an example I/O interface that enables computer system 1000 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Wash., U.S.A.).

The communication between I/O interfaces 1010 and external devices 1012 can occur across wired and/or wireless communications link(s) 1011, such as Ethernet-based wired or wireless connections. Example wireless connections include cellular, Wi-Fi, Bluetooth®, proximity-based, near-field, or other types of wireless connections. More generally, communications link(s) 1011 may be any appropriate wireless and/or wired communication link(s) for communicating data.

Particular external device(s) 1012 may include one or more data storage devices, which may store one or more programs, one or more computer readable program instructions, and/or data, etc. Computer system 1000 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 1000 may be operational with numerous other general purpose or special purpose computing system environments or configurations. Computer system 1000 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects described herein may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples. For example, computing environments of other architectures can be used to incorporate and use one or more embodiments.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
receiving, via a user interface of a user device, one or more user-provided inputs, the one or more user-provided inputs including at least one indication that a user is exhibiting one or more symptoms of an illness;
establishing, based on at least some of the received user-provided inputs, an advisory process preconfigured to assist the user in treating, using a plurality of specific medications, symptoms of the illness by, at least in part, providing electronic reminders to the user according to one or more predefined time intervals, each reminder of the electronic reminders indicating that the user is to take a respective one or more specific medications, of the plurality of specific medications, to treat the one or more symptoms of the illness;
providing to the user, based on the established advisory process, a plurality of electronic reminders to take the respective one or more specific medications to treat the one or more symptoms of the illness and being exhibited by the user, the plurality of electronic reminders being provided according to the one or more predefined time intervals; and
tracking one or more acknowledgments, received from the user, that the user has taken at least one of the one or more specific medications, indicated by one or more of the plurality of electronic reminders, to treat the one or more symptoms of the illness.

2. The computer-implemented method of claim 1, wherein the one or more predefined time intervals are either (i) preconfigured as part of the advisory process, or (ii) defined by the user as an input of the one or more user-provided inputs.

3. The computer-implemented method of claim 1, wherein the one or more predefined time intervals (i) align with intended dose times that the user is to take the respective one or more specific medications, or (ii) align with a user-defined amount of time prior to the intended dose times that the user is to take the respective one or more specific medications.

4. The computer-implemented method of claim 1, further comprising periodically issuing, during the advisory process, a prompt asking for symptom updates, the prompt instructing the user is to indicate any symptoms of the illness that the user is still exhibiting.

5. The computer-implemented method of claim 1, further comprising receiving from the user a selection that indicates for which of the respective one or more specific medications, of the plurality of specific medications, the user would like to receive the plurality of electronic reminders.

6. The computer-implemented method of claim 1, wherein the preconfigured advisory process includes a timeout for automatically dismissing a reminder of the plurality of electronic reminders provided to the user based on determining that no acknowledgment that the user has taken a dose of medication corresponding to the reminder has been received from the user, wherein the automatically dismissing issues an instruction to the user to skip that dose of medication corresponding to the reminder.

7. The computer-implemented method of claim 6, wherein the issued instruction indicates to the user to resume taking the respective one or more specific medications at a next-scheduled dose time.

8. The computer-implemented method of claim 1, wherein the plurality of specific medications, of the preconfigured advisory process, include at least four specific medications.

9. The computer-implemented method of claim 1, wherein the plurality of specific medications, of the preconfigured advisory process, is selected from the group consisting of a homeopathic medication, an antihistamine, a decongestant, and a pain reliever.

10. The computer-implemented method of claim 1, wherein the method further comprises:
recording, as a user record, (i) at least one of the one or more user-provided inputs, the user record of the one or more user-provided inputs indicating any of the one or more symptoms of the illness the user is continuing to experience during a time during which the advisory process is being followed, and (ii) at least one of the one or more acknowledgments being tracked, the user record of the one or more acknowledgments indicating a dose history; and
providing, based on the recording, the user record to the user, the user record being accessible by the user to share with one or more individuals.

11. A kit for treating symptoms of an illness, the kit comprising:
a plurality of medications, each medication of the plurality of medications configured for treating a respective one or more symptoms of the symptoms of the illness, and each medication of the plurality of medications being separately-dosed, the plurality of medications comprising:
a homeopathic medication;
an antihistamine;
a decongestant; and
a pain reliever; and
an instructional insert comprising a treatment strategy for treating the symptoms of the illness, the treatment strategy providing a multi-day dosage regimen indicating when to individually take each medication of the plurality of medications.

12. The kit of claim 11, wherein the plurality of medications are single-active-ingredient medications, and wherein the plurality of medications consists of the homeopathic medication, the antihistamine, the decongestant, and the pain reliever.

13. The kit of claim 11, wherein the homeopathic medication includes a zinc compound.

14. The kit of claim 11, wherein the antihistamine is a first-generation antihistamine.

15. The kit of claim 11, wherein the antihistamine includes Chlorpheniramine Maleate.

16. The kit of claim 11, wherein the decongestant includes Phenylephrine HCl.

17. The kit of claim 11, wherein the pain reliever includes Analgesic-Antipyretic, Naproxen Sodium.

18. The kit of claim 11, wherein the multi-day dosage regimen includes (i) a first phase of a set duration for taking the homeopathic medication, the antihistamine, the decongestant, and the pain reliever, and (ii) a second phase of a variable duration for taking only the decongestant and the pain reliever, the variable duration being variable dependent on whether the symptoms of the illness persist.

19. The kit of claim 11, wherein the kit comprises a quantity of each medication, of the plurality of medications, to resolve the symptoms of the illness within 168 hours.

20. The kit of claim 19, wherein the quantity comprises:
at least 225 mg of the homeopathic medication;
between 30 mg and 50 mg of the antihistamine;
between 100 mg and 400 mg of the decongestant; and
between 1200 mg and 4,000 mg of the pain reliever.
